(19) United States
(12) Patent Application Publication
(10) Pub. No.: US 2005/0019828 A1
(43) Pub. Date: Jan. 27, 2005

(54) Gelatin coated receiver as protein microarray substrate

(76) Inventors: Tiecheng A. Qiao, Webster, NY (US); Thomas L. Penner, Fairport, NY (US); Howard W. Chou, Pittsford, NY (US); George W. Roth, Lima, NY (US)

Correspondence Address: Paul A. Leipold;Patent Legal Staff, Eastman Kodak Company, 343 State Street, Rochester, NY, 14650-2201, US

(21) Appl. No.: 10/625,646

(22) Filed: Jul. 23, 2003

(51) Int. Cl. G01N 33/53; C12M 1/34; G01N 33/543

(52) U.S. Cl. 435/7.1; 435/287.2; 436/518

(57) ABSTRACT

A protein microarray element comprising: a) a support; b) a gelatin layer containing functional groups capable of binding biological probes; and interposed between the support and the gelatin layer and c) an adhesive interlayer layer capable of maintaining contact with the support and with the gelatin layer.

### FIELD OF THE INVENTION

The present invention relates to fabricating protein microarrays in general, and in particular to a method that utilizes a gelatin-based substrate wherein the gelatin surface is modified to improve specific attachment of biological molecules.

### BACKGROUND OF THE INVENTION

The completion of Human Genome project spurred the rapid growth of a new interdisciplinary field of proteomics which includes: identification and characterization of complete sets of proteins encoded by the genome, the synthesis of proteins, post-translational modifications, as well as detailed mapping of protein interaction at the cellular regulation level.

While 2-dimensional gel electrophoresis in combination with mass spectrometry still remains the dominant technology in proteomics study, the successful implantation and application of DNA microarray technology to gene profiling and gene discovery have prompted scientists to develop protein microarray technology and apply microchip based protein assays to the field of proteomics. For example, in WO 00/04382 and WO 00/04389, a method of fabricating protein microarrays is disclosed. A key element in the disclosure is a substrate consisting of a solid support coated with a monolayer of thin organic film on which protein or a protein capture agent can be immobilized.

Nitrocellulose membrane was widely used as a protein blotting substrate in Western blotting and enzyme linked immunosorbent assay (ELISA). In WO 01/40312 and WO 01/40803, antibodies are spotted onto a nitrocellulose membrane using a gridding robot device. Such spotted antibody microarrays-on a nitrocellulose membrane substrate have been shown to be useful in analyzing protein mixture in a large parallel manner.

In WO 98/29736, L. G. Mendoza et al. describe an antibody microarray with antibody immobilized onto a N-hydroxysuccinimidyl ester modified glass substrate. In U.S. Pat. No. 5,981,734 and WO 95/04594, a polyacrylamide based hydrogel substrate technology is described for the fabrication of DNA microarrays. More recently, in Anal. Biochem. (2000) 278, 123-131, the same hydrogel technology was further demonstrated as useful as a substrate for the immobilization of proteins in making protein microarrays.

In the above cited examples, the common feature among these different approaches is the requirement of a solid support that allows covalent or non-covalent attachment of a protein or a protein capture agent on the surface of said support. In DNA microarray technology, a variety of surfaces have been prepared for the deposition of pre-synthesized oligos and PCR prepared cDNA probes. For example, in EP 1 106 603 A2, a method of preparing vinylsulfonyl reactive groups on the surface to manufacture DNA chip is disclosed. Even though the invention is useful in preparing DNA chip, it is not suitable for protein microarray applications. Unlike DNA, proteins tend to bind to surfaces in a non-specific manner and, in doing so, lose their biological activity. Thus, the attributes for a protein microarray substrate are different from those for a DNA microarray substrate in that the protein microarray substrate must not only provide surface functionality that are capable of interacting with protein capture agents, but must also resist non-specific protein binding to areas where no protein capture agents have been deposited.

Bovine serum albumin (BSA) has been demonstrated to be a useful reagent in blocking proteins from non-specific surface binding. Polyethylene glycol and phospholipids have also been used to passivate surfaces and provide a surface resistant to non-specific binding. However, all of these methods suffer disadvantages either because surface preparation takes a long time or because the method of surface modification is complex and difficult, making the method less than an ideal choice for large scale industrial manufacture.

U.S. applications Ser. Nos. 10/020,747 and 10/091,644, describe a low cost method of making protein microarray substrate using gelatin coating to create a reactive surface for immobilization of protein capture agents. While the gelatin modified surface effectively eliminates non-specific protein binding, it is desirable to coat such a surface onto a dimensional stable solid support for the convenience of use.

The art needs a dimensionally stable substrate with chemical functionality for the immobilization of protein capture agents, but such substrate must have enough adhesive strength on its surface to bind the upper layer of the coated gelatin so that the gelatin layer does not frill when the coated substrate is wet during any biological processing and does not strip when the coated substrate is dry.

Glass plate is known in the art to be dimensionally stable and is a preferred solid support for biological uses. Coating a hydrophilic binder, e.g. gelatin, onto glass is a very demanding task because a compatible adhesive interlayer must be applied between glass and the binder. Such adhesive interlayer should have the following properties: 1. it must be a thin film that does not have optical interferences for the protein microarray applications; 2. it must not contain any components that chemically interfere with the protein capture agent attachment chemistry incorporated onto binder surface; 3. it must be readily manufactured.

It is the purpose of this invention to provide a protein microarray element that solves some of the problems discussed above and also to provide a method of making such a protein microarray element.

### SUMMARY OF THE INVENTION

The present invention overcomes some of the problems discussed above by providing a protein microarray element comprising:
a) a support;
b) a gelatin layer containing functional groups capable of binding biological probes; and interposed between the support and the gelatin layer
c) an adhesive interlayer capable of maintaining contact with the support and with the gelatin layer.

Another embodiment of the invention discloses a protein microarray element comprising:
a) a support;
b) on said support is disposed an adhesive layer capable of maintaining contact with the support and with; and
c) a gelatin layer that bears a trifunctional compound A-L-B; wherein A is a functional group capable of interacting with the gelatin; L is a linking group capable of interacting with A and with B; and B is a functional group capable of interacting with a protein capture agent; wherein A may be the same or different from B.

In yet another embodiment of the invention, there is disclosed a method of making a gelatin-based substrate for fabricating protein arrays, comprising the steps of:
providing a support;
coating on the support an adhesion layer;
coating, on said adhesion layer, a layer of gelatin containing a trifunctional compound A-L-B; wherein A is a functional group capable of interacting with the gelatin; L is a linking group capable if interacting with A and with B; and B is a functional group capable of interacting with a protein capture agent;
   wherein A may be the same or different from B.

The invention is particularly useful in fabricating protein microarrays. The invention provides a substrate with functionalities capable of interacting specifically with protein capture agents that are immobilized on its surface; and also the substrate substantially resistant to non-specific binding.

When compared with unmodified gelatin substrates, the substrates prepared according to the present invention can detect analytes even when they are in very low concentrations in the biological sample. The gelatin substrates of the invention can be readily manufactured at low cost. The usefulness of the claimed substrate for protein attachment is demonstrated below in the examples, using several chemical modification methods and enzyme linked immunosorbent assay (ELISA).

### DETAILED DESCRIPTION OF THE INVENTION

In general, a protein microarray of the invention can be prepared by first modifying a solid support, namely the protein microarray support, followed by depositing various protein capture agents onto the modified substrate at pre-defined locations. Supports of choice for protein microarray applications can be organic or inorganic. Some commonly used support materials include glass, plastics, metals, and semiconductors. The support can be transparent or opaque, flexible or rigid. In some cases, the support can be a porous membrane e.g. nitrocellulose and polyvinylidene difluoride, and the protein capture agents are deposited onto the membrane by physical adsorption. However, to improve robustness and reproducibility, it is more desirable to use a solid support that has dimensional stability.

Glass, or fused silica, is the most commonly used microarray support in the art. A conventional way of generating protein attachment chemistry on a glass surface is to use silane coupling chemistry as described by Edwin P. Plueddemann, “Silane Coupling Agents” 2nd Ed., Plenum Press, New York, 1991, to graft the appropriate protein attachment chemistry onto a glass surface. To perform such grafting, a glass surface must be either plasma discharge treated or chemically treated with chemical reagents to provide a hydrophilic surface. However, it is very difficult to generate a highly uniform and defect-free surface using these treatments. Moreover, these treatments can not be easily integrated with a coating method that renders a low cost and manufacturable means of making a protein microarray substrate.

Generally, a glass support is planar, and it has high flatness and clarity. Preferably, the glass does not fluoresce, and is from 0.1 mm to 5 mm, preferably from 0.5 to 2.0 mm in thickness. The glass support can be any dimensions and can be cut into various sizes according to its intended uses. In a preferred embodiment of this invention, a glass surface is coated with an interlayer to provide a hydrophilic surface for the subsequent coating of upper gelatin layer that is incorporated with the protein capture agents immobilization chemistry.

Gelatin has been used in the photographic industry as a binder for various chemical components, and the process of making high quality gelatin is well established in the industry. Because gelatin is made of biological materials, it is biologically compatible with protein capture agents on the protein microarray. The gelatin coated surface provides a biologically benign surface for the immobilization of protein capture agents onto the protein microarray. As shown in this invention, gelatin also renders a surface that substantially reduces background noise that is a result of non-specific binding.

Normally, gelatin is coated onto a support and gelation occurs through a process by which gelatin solutions or suspensions of gelatin and other materials form continuous three-dimensional networks that exhibit no steady state flow. This can occur in polymers by polymerization in the presence of polyfunctional monomers, by covalent cross-linking of a dissolved polymer that possesses reactive side chains and by secondary bonding, for example, hydrogen bonding, between polymer molecules in solution. Polymers such as gelatin exhibit thermal gelation which is of the latter type. The process of gelation or setting is characterized by a discontinuous rise in viscosity. (See, P. I. Rose, “The Theory of the Photographic Process”, 4^{th} Edition, T. H. James ed. pages 51 to 67).

When gelatin is coated on a solid support, e.g. glass, plastic, metal, an interlayer is necessary to prevent frilling of the gelatin coating when the upper gelatin coating is wet during any biological processing and to prevent stripping when the upper gelatin coating is dry. Generally, an interlayer consists of film forming hydrophilic colloidal materials or hydrophilic binders.

In addition to providing adequate adhesive force for binding the gelatin layer, the interlayer should also be optically transparent and not fluoresce. Typical interlayer materials include, but are not limited to, naturally occurring substances such as proteins, protein derivatives, gelatin (e.g., alkali-treated gelatin such as cattle bone or hide gelatin, or acid treated gelatin such as pigskin gelatin), and gelatin derivatives (e.g., acetylated gelatin, phthalated gelatin, and the like). Also useful as vehicle extenders are hydrophilic water-permeable colloids. These include synthetic polymeric peptizers, carriers, and/or binders such as poly(vinyl alcohol), poly(vinyl lactams), acrylamide polymers, polyvinyl acetals, polymers of alkyl and sulfoalkyl acrylates and methacrylates, hydrolyzed polyvinyl acetates, polyamides, polyvinyl pyridine, methacrylamide copolymers, and the like.

In the case of gelatin as the preferred interlayer material, an organic solvent, or a mixture of solvents, should also be included in the formulation. Examples of such organic solvent include, but not limited to, acetone, alcohol, ethyl acetate, methylene chloride, ether, or a mixture of the foregoing. In order to uniformly mix gelatin with these organic solvent, a dispersing aid can be, but not necessary, added to the formulation, e.g. organic acids or bases. To improve adhesive strength of the interlayer, silicate salt, e.g. sodium silicate, is also included in the interlayer formulation. To improve the physical strength of the interlayer, it is preferred that gelatin in the interlayer is hardened using one or more than one crosslinking agent. Examples of gelatin hardening agents can be found in standard references such as The Theory of the Photographic Process, T. H. James, Macmillan Publishing Co., Inc. (New York 1977) or in Research Disclosure, September 1996, Number 389, Part IIB (hardeners). Inorganic hardening agents are preferred over organic hardeners.

In another embodiment of the invention, a polymeric support can be used to coat the gelatin layer that is incorporated with protein capture agent immobilization chemistry. Typical polymeric supports which form supporting surfaces according to this invention include cellulose esters such as cellulose nitrate and cellulose acetate; poly(vinyl acetal) polymers, polycarbonates, polyesters such as polymeric, linear polyesters of bifunctional saturated and unsaturated aliphatic and aromatic dicarboxylic acids condensed with bifunctional polyhydroxy organic compounds such as polyhydroxy alcohols—e.g. polyesters of alkylene glycol and/or glycerol with terephthalic, isophthalic, adipic, maleic, fumaric and/or azelaic acid; polyhalohydrocarbons such as polyvinyl chloride; and polymeric hydrocarbons, such as polystyrene and polyolefins, particularly polymers of olefins having from 2 to 20 carbon atoms. These supports may be used alone or may be utilized as coatings on metal, glass, and other solid surface. It is preferred that the support has substantial dimensional stability when wet.

When polymer support is used, surface treatment is necessary to render the appropriate adhesiveness for binding the gelatin layer. For example, discharge treatment, flame treatment, ultraviolet ray treatment, high frequency treatment, active plasma treatment, laser treatment, glow discharge, LTV exposure, electron beam treatment or the like as described in U.S. Pat. Nos. 2,764,520, 3,497,407, 3,145,242, 3,376,208, 3,072,483, 3,475,193, 3,360,448, British Pat. No. 788,365, etc., can be used. Polymer supports can be surface-treated with adhesion-promoting agents including dichloroacetic acid and trichloroacetic acid, phenol derivatives such as resorcinol and p-chloro-m-cresol, solvent washing prior to overcoating with a subbing interlayer, e.g. the gelatin interlayer described above. In addition to surface treatment or treatment with adhesion promoting agents, additional adhesion promoting primer or tie layers containing polymers such as vinylidene chloride-containing copolymers, butadiene-based copolymers, glycidyl acrylate or methacrylate-containing copolymers, maleic anhydride-containing copolymers, condensation polymers such as polyesters, polyamides, polyurethanes, polycarbonates, mixtures and blends thereof, and the like may be applied to the polyester support. Particularly preferred primer or tie layers comprise a chlorine containing latex or solvent coatable chlorine containing polymeric layer. Vinyl chloride and vinylidene chloride containing polymers are preferred as primer or subbing layers of the present invention.

It has been recognized in the art, as described in U.S. Pat. Nos. 3,864,132, and British Pat. No. 1,066,944, that a hydrophilic colloid layer can be firmly bonded to a hydrophobic polymer supporting surface by means of an inorganic oxide adhesive layer which is contiguous to the supporting surface and to the hydrophilic colloid layer. Such adhesive layers (commonly referred to as subbing layers in the arts) are binderless layers which consist essentially of inorganic metal oxide and are capable of bonding directly and tenaciously to both hydrophilic colloid layers and to hydrophobic polymeric support surfaces to perform the function heretofore performed by considerably more complex polymer layers. The term “binderless layer” refers to a layer that is substantially free of organic adhesive materials and refers particularly to the absence of those organic adhesive and binder materials commonly utilized in the arts, such as natural and synthetic polymeric binders and colloidal vehicles. The binderless adhesive layer may be formed of crystalline or amorphous inorganic oxides. Oxides of silicon, such as silicon monoxide and silicon dioxide, are preferred inorganic oxides, since they are substantially water insoluble and chemically inert in photographic processing and use environments and are essentially transparent. Silicon oxides are also preferred since they can be vapor deposited by heating to vaporization temperatures that are low as compared to those required for vaporizing the other inorganic oxides utilized in the practice of this invention. Aluminum oxide, boron-silicon oxide, magnesium oxide, tantalum oxide and titanium oxide as well as mixtures thereof are particularly suited to the practice of this invention. The inorganic oxide adhesive layer may be utilized on glass support.

Furthermore, an adhesive interlayer as described in U.S. Pat. Nos. 3,511,661, and 3,860,426, can be used on metal support. For instance, aluminum is a preferred metal support in lithographic plate industry due to its availability and low cost. Generally an anodic oxidation as described in U.S. Pat. Nos. 4,608,131, 4,092,169, and 4,446,221, is carried out on aluminum support surface before the application of the adhesive interlayer.

To immobilize protein capture agents, the support coated with an adhesive interlayer needs to be further coated with a layer of gelatin modified by certain chemical functional agents. In general, the chemically functional agent is a bi-functional molecule which can be represented as A-L-B, in which A and B are chemical functionalities that are capable of reacting or interacting with gelatin and protein capture agent molecules to be immobilized on the substrate and L is linkage group. Preferably, L is a di-radical of such a length that the shortest through-bond path between the ends that connect A to B is not greater than 10 atoms.

There are two classes of bi-functional agents: 1). homofunctional agent if A=B; and 2). heterofunctional agent if A # B. Some commonly used A and B include but are not limited to, aldehyde, epoxy, hydrazide, vinyl sulfone, succinimidyl ester, carbodiimide, maleimide, dithio, iodoacetyl, isocyanate, isothiocyanate, aziridine. The linking group L comprises any reasonable combination of relatively non-labile covalently bonded chemical units sufficient to connect the two functionalities A and B. These chemical units can consists of, but are not necessarily limited to, a single bond, a carbon atom, an oxygen atom, a sulfur atom, a carbonyl group 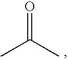
a carboxylic ester group 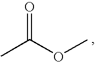
a carboxylic amide group 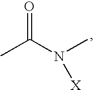
a sulfonyl group 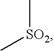
a sulfonamide group 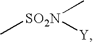
an ethyleneoxy group, a polyethyleneoxy group, or an amino group 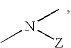
where substituents X, Y, and Z are each independently a hydrogen atom, or an alkyl group of 1-10 carbon atoms; and linear or branched, saturated or unsaturated alkyl group of 1 to 10 carbon atoms (such as methyl, ethyl, n-propyl, isopropyl, t-butyl, hexyl, decyl, benzyl, methoxymethyl, hydroxyethyl, iso-butyl, and n-butyl); a substituted or unsubstituted aryl group of 6 to 14 carbon atoms (such as phenyl, naphthyl, anthryl, tolyl, xylyl, 3-methoxyphenyl, 4-chlorophenyl, 4-carbomethoxyphenyl and 4-cyanophenyl); and a substituted or unsubstituted cycloalkyl group of 5 to 14 carbon atoms such as cyclopentyl, cyclohexyl, and cyclooctyl); a substituted or unsubstituted, saturated or unsaturated heterocyclic group (such as pyridyl, primidyl, morpholino, and furanyl); a cyano group. Some solubilizing groups can also be introduced into A-L-B and examples of these solubilizing groups include, but are not limited to, carboxylic acid, sulfonic acid, phosphonic acid, hydroxamic acid, sulfonamide, and hydroxy groups (and their corresponding salts). A and B can also be in the form of readily reactive functionalities towards crosslinkers, examples include but not limited to carboxy, amino, and chloromethyl, etc. A and B can be affinity tags that are capable of interacting non-covalently with the protein capture agents intended to be immobilized onto the substrate. For example, some commonly used tag systems include, but are not limited to, streptavidin and biotin, histidine tags and nickel metal ions, glutathione-S-transferase and glutathione. One skilled in the art should be able to create a fusion protein capture agent using recombination DNA technology and an element of tag recognition unit can be introduced into protein capture agent in this way.

The present invention is designed to attain very high densities of chemical moieties that are useful in the immobilization of proteins. To accomplish this, the invention employs a “polymer scaffold” strategy. For the purposes of this invention, the term “polymer scaffold” refers to a linear or branched polymer, rich in specific functionalities, that extends out in a 3-dimensional fashion from a surface. In this case, functional groups consist of chemical units capable of immobilizing proteins and the surface is protein. In one basic strategy for the preparation of a protein-receptive polymer scaffold, a precursor polymer is utilized which is rich in units that are capable of being converted into chemical functions that will immobilize proteins. The precursor polymer is affixed to a gelatin surface and then converted to a protein-receptive form by post-treatment with a chemical agent. By “affixed” it is meant that the precursor polymer is applied to the gelatin surface and adheres to the gelatin by any of a number of chemical and physical attractive mechanisms including ionic interactions, covalent bonds, coordinative bonds, hydrogen bonds, and Van-der-Waals interactions.

In a preferred embodiment, the chemical agent will be one of the A-L-B structures defined above and the precursor polymer will be rich in such reactive units as thiols, amines, phosphines, alcohols, or carboxylic acids. Preferably the reactive unit is a primary or secondary amine. Specific polymers which can be used for this purpose may be selected from the set consisting of, but not necessarily limited to poly (propyleneimine) and polymers and copolymers of N-aminopropyl (meth)acrylamide and secondary amine derivatives thereof, N-aminoethyl (meth)acrylate and secondary amine forms thereof, diallyamine, vinylbenzylamine, vinylamine, (meth)acrylic acid, vinylbenzyl mercaptan, and hydroxyethyl(meth)acrylate. Preferably, the polymer is poly(vinylamine), poly(propyleneimine), or poly(N-aminopropyl methacrylamide).

The affixing of the scaffold polymer to the surface of the gelatin can be achieved using any chemical agent or technique that is known to result in the formation of a covalent bond between the reactive units of the polymer and either the amine or carboxylic acid functionality of the gelatin. For example, a dehydrating agent such as a carbodiimide, a pyridyl dication ether, or a carbamoylpyridinium compound can be used to bind an amine-containing polymer or a carboxylic acid-containing polymer to a gelatin surface via amide bonds. Similarly, a bis(vinylsulfonyl) compound can be used to bind poly(ethyleneimine) to a gelatin surface. Once the scaffold polymer is affixed to the gelatin surface, it is then treated with an excess of the appropriate A-L-B compound to afford the reactive surface with a high level of reactive units.

A second basic strategy for the preparation of a protein-receptive polymer scaffold involves the direct affixing onto the gelatin surface of a polymer rich in chemical functions that will immobilize proteins. Such functions include, but are not necessarily limited to include but are not limited to: aldehyde, epoxy, hydrazide, vinyl sulfone, succinimidyl ester, carbodiimide, maleimide, dithio, iodoacetyl, isocyanate, isothiocyanate, and aziridine. Additionally, more than one type of polymer scaffold polymer may be affixed to the same gelatin substrate.

Formula I represents a preferred polymer for forming the polymer scaffold: 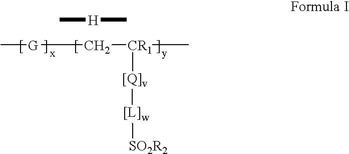
wherein R₁ is a hydrogen atom or a C₁-C₆ alkyl group. Preferably R1 is a hydrogen atom.
Q is —CO₂—, or CONR₁.;
v is 1 or 0;
w is 1-3;
L is a divalent linking group containing at least one linkage selected from the group consisting of —CO₂— and —CONR₁, and containing 3-15 carbon atoms, or a divalent atom containing at least one linkage selected from the group consisting of —O—, —N(R₁)—, —CO—, —SO—, —SO₂—, —SO₃—, —SO₂N(R₁)—, —N(R₁)CON(R₁)— and —N(R )CO₂—, and containing 1-12 carbon atoms in which R₁ has the same meaning as defined above;
R₂ is —CH═CH2 or —CH2-CH2X, wherein X₁ is a substituent replaceable by a nucleophilic group or releasable in the form of HX₁ by a base. X₁ may be, but is not necessarily limited to —S₂O₃⁻, —SO₄⁻, —Cl, —Br, —I, quaternary ammonium, pyridinium, and —CN, and sulfonate esters (such as mesylate and tosylate); x and y both represent molar percentages ranging from 10 to 90 and 90 to 10. Preferably, x and y range from 25 to 75 and 75 to 25, respectively.

In a preferred embodiment of this invention, a polymer containing pendant vinylsulfone or vinylsulfone precursor units may be reacted with the gelatin in order to provide a polymer scaffold. Polymers preferred for this embodiment are represented by the structure in formula 1, and consist of the polymerization products of a “G” monomer, which affords to polymer with favorable solubility properties, and a “H” monomer, which contains the vinylsulfone moiety or, more preferably, a vinylsulfone precursor function, such as a sulfonylethyl group with a leaving group in the β-position. More than one type each of G and H monomers may be present in the same polymer. Although the polymer may have any molecular weight, molecular weights (Mn) between 1000 and 200,000 AMU are preferred. Molecular weights between 2000 and 50,000 AMU are especially preferred provided that the polymer is soluble in water or a mixture of water and water-miscible solvents (such as methanol, ethanol, acetone, tetrahydrofuran, etc.). Additional monomers can be incorporated in order to modify properties such as glass transition temperature, surface properties, and compatibility with other formulation components as needed for specific applications. Selection of additional monomers will be application dependent and will be obvious to one skilled in the art.

G is a polymerized α,β-ethylenically unsaturated addition polymerizeable monomer which imparts water-solubility to the polymer. The monomer from which G can be derived include both ionic and nonionic monomers. Ionic monomers may include, for example, anionic ethylenically unsaturated monomers such as 2-phosphatoethyl acrylate potassium salt, 3-phosphatopropyl methacrylate ammonium salt, acrylamide, methacrylamides, maleic acid and salts thereof, sulfopropyl acrylate and methacrylate, acrylic and methacrylic acids and salts thereof, N-vinylpyrrolidone, acrylic and methacrylic esters of alkylphosphonates, styrenics, acrylic and methacrylic monomers containing amine ammonium functionalities, styrenesulfonic acid and salts thereof, acrylic and methacrylic esters of alkylsulfonates, vinylsulfonic acid and salts thereof. Nonionic monomers may include monomers containing hydrophilic, nonionic units such as poly(ethylene oxide) segments, carbohydrates, amines, amides, alcohols, polyols, nitrogen-containing heterocycles, and oligopeptides. Examples include, but are not limited to poly(ethylene oxide) acrylate and methacrylate esters, vinylpyridines, hydroxyethyl acrylate, glycerol acrylate and methacrylate esters, (meth)acrylamide, and N-vinylpyrrolidone.

Preferably, G is the polymerized form of acrylamide, sodium 2-acrylamido-2-methanepropionate, sulfopropyl acrylate and methacrylate salts, or sodium styrenesulfonate.

Monomer H is the polymerized form of a vinylsulfone or vinylsulfone precursor unit covalently bound to a polymerizeable α,β-ethylenically unsaturated function by an organic spacer which consists of Q and L, of which Q is an optional component.

Vinylsulfone and vinylsulfone-containing precursor “H” monomers useful in this embodiment include, but are not necessarily limited to those compounds disclosed in U.S. Pat. Nos. 4,548,869 and 4,161,407 (incorporated herein by reference) as well as those compounds in Formula II.

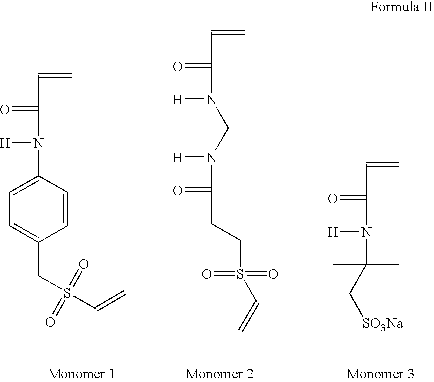

Although the polymer may have any molecular weight, molecular weights (Mn) between 1000 and 200,000 AMU are preferred. Molecular weights between 2000 and 50,000 AMU are especially preferred.

Once a solid support, the corresponding interlayer formulation, and the gelatin binding layer formulation are selected, it is preferred that the interlayer and the upper gelatin later may be applied to the solid support using an in-line process during the microarray substrate manufacture. However, it may also be made in separate processes. The interlayer and the upper gelatin layer can be coated on the support using the methods that is broadly described by Edward Cohen and Edgar B. Gutoff in Chapter 1 of “Modem Coating And Drying Technology”, (Interfacial Engineering Series; v.1), (1992), VCH Publishers Inc., New York, N.Y. To achieve ultra thin film coating with the interlayer application, it is desirable that the interlayer is coated using either gravure method, as described in U.S. Pat. Nos. 3,283,712, 3,468,700, and 4,325,995, or wicked coating method, as described in U.S. Pat. Nos. 3,000,349, 3,786,736, 3,831,553, and 4,033,290.

The gelatin layer described in this invention can either be coated as is on any solid support, or with one or a combination of multiple hardening agents mixed in the gel. The level of the hardening agent should be from 0 to 20 wt. %, and preferably 0.5 to 8 wt. %, of the total gelatin coated.

There are two types of gelatin: acid pretreated and alkaline pretreated. The preferred gelatin is alkaline pretreated gelatin from bovine bone marrow, but gelatin can also come from other sources. Examples include, but are not limited to, pig gelatin, fish gelatin. The bi-functional agent A-L-B can be introduced either during or after the gelatin coating onto a solid support.

In general, a fluid coating composition contains a binder, a solvent to dissolve or suspend the components, and optional additives such as surfactants, dispersants, plasticizers, biocides, cross-linking agents for toughness and insolubility, and conductive materials to minimize static buildup. All the components are mixed and dissolved or dispersed, and the coating fluid is sent to an applicator where it is applied to a substrate by one of several coating techniques. Heat is then applied to the coating to evaporate the solvent and produce the desired film, or the coating is solidified by the action of ultraviolet radiation or an electron beam.

The most suitable coating method—including the coating speed—will depend on the quality and functionality desired and the materials being used, e.g., the substrate, the solvent, weight and viscosity of the coating, etc. For a single layer format, suitable coating methods may include dip coating, rod coating, knife coating, blade coating, air knife coating, gravure coating, forward and reverse roll coating, and slot and extrusion coating.

Coating speed can also be an important determinant in the choice of coating method. Although most methods can be used at low speeds, and all methods have a limiting upper speed, some work better at higher speeds. Curtain coating requires a minimum flow to maintain the integrity of the curtain. Therefore, this method is limited to higher speeds id a thin coating is to be obtained. In slide coating of multiple layers, interfacial instabilities are more likely to occur on the slide when the layers are very thin. Higher speeds, with their higher flows and thicker layers on the slide, tend to avoid these instabilities. See, p. 12, “Modern Coating and Drying Technology”, supra.

The gelatin has a laydown of 0.2 to 100 grams per square meter; preferably 10 to 50 grams per square meter.

Any well known coating method, such as bead coating or curtain coating, can be used to prepare the gelatin substrate. The gelatin could be coated with any other coating aids such as surfactants and thickeners to adjust its physical property. The gelatin used in the invention may be chemically modified either before, during or after the coating process to create more chemical functionalities that can react or interact with biologically active molecules or assemblies intended to be attached on this substrate.

In general, there are two ways to prepare a reactive surface for protein capture agent immobilization using gelatin coating method. In the first approach, the chemical agent or polymer scaffold can be mixed with gelatin with certain coating aids and the mixture is coated on a solid support as described. In the second approach, a gelatin coating is prepared on a solid support as described above, and upon drying, the gelatin coating is dipped into a solution containing chemical agents, e.g. A-L-B, polymer scaffold, to affix the reactive chemistry to the gelatin surface. Alternatively the attachment chemistry can also be coated on a gelatin surface. It is preferred that the polymer scaffold is introduced to the substrate surface during gelatin coating to simplify the manufacture process.

Once a protein microarray substrate is modified with the polymer scaffold, protein capture agents will be placed onto the substrate to generate protein microarray content. A protein molecule consists of 20 amino acids that are connected in linear manner covalently. Some proteins can be further modified at selected amino acids through posttranslational processes that include phosphorylation and glycosylation. A protein molecule can be used as a protein capture agent. As used herein, the term “protein capture agent” means a molecule that can interact with proteins in high affinity and high specificity. Typically it is desirable to have an affinity binding constant between a protein capture agent and target protein greater than 10⁶ M⁻¹. There are several classes of molecules that can be used as protein capture agents on a protein microarray. Antibodies are a class of naturally occurring protein molecules that are capable of binding targets with high affinity and specificity. The properties and protocols of using antibody can be found in “Using Antibodies; A Laboratory Manual”, (Cold Spring Harbor Laboratory Press, by Ed Harlow and David Lane, Cold Spring Harbor, N.Y. 1999). Antigens can also be used as protein capture agents if antibodies are intended targets for detection. Protein scaffolds such as whole protein/enzyme or their fragments can be used as protein capture agents as well. Examples include phosphotases, kinases, proteases, oxidases, hydrolyases, cytokines, or synthetic peptides. Nucleic acid ligands can be used as protein capture agent molecules after in vitro selection and enrichment for their binding affinity and specificity to certain targets. The principle of such selection process can be found in Science, Vol. 249, 505-510, 1990 and Nature, Vol. 346, 818-822, 1990. U.S. Pat. No. 5,110,833 discloses an alternative class of synthetic molecules that can mimic antibody binding affinity and specificity and can be readily prepared by the so called Molecular Imprinting Polymer (MIP). This technology has been reviewed in Chem. Rev. Vol. 100, 2495-2504, (2000).

In practice, a protein microarray is brought into contact with a biological fluid sample, proteins in the sample will adsorb to both areas spotted with specific protein capture agents and areas without protein capture agents. Since the protein microarray is intended to be used for the measurement of specific interactions between protein capture agents on the chip with certain proteins or other molecules in the biological fluid sample, the non-specific binding of sample proteins to non-spotted area would give rise to high background noise. The term non-specific binding refers to the tendency of protein molecules to adhere to a solid surface in a non-selective manner. This high background noise resulting from the non-specific binding will interfere with reporter signals to be detected from the spotted area unless the non-specific binding is blocked in an appropriate manner. Typically, the protein microarray will be immersed in a solution containing a blocking agent to block the non-specific binding sites before its contact with the intended analyte solution. A commonly used method for blocking protein non-specific binding is to treat the surface of the substrate with a large excess of bovine serum albumin. The non-spotted surface area may also be chemically modified with polyethylene glycol (PEG), phospholipid, or poly lysine to prevent non-specific binding.

The invention can be better appreciated by reference to the following specific embodiments.

### EXAMPLES

### Example 1

This example illustrates the formulation of interlayer and upper gelatin melts and the method of coating the melts onto a glass support. It illustrates the usefulness of using an interlayer to provide the needed adhesiveness of binding gelatin onto a glass surface.

Formulation 1-1 (Gelatin Melt):

Solution 1 :This was prepared by adding 726.54 grams of swollen Type IV gelatin (24.8% w/v) in 2237.06 grams of water, 16 grams of coating aid of Nonylphenoxypolyglycerol, 20.4 grams of coating aid Sodium octyl phenol poly (etheneoxy) sulfonate.

Solution 2: This was prepared by adding 800.79 grams of Ethene, 1,1′-(methylenebis(sulfonyl))bis (1.8% w/v) and 2199 grams of distilled water.

Solution 1 and solution 2 are mixed in equal volume to make into a single melt before coating.

Formulation 1-2 (Interlayer Melt):

This was prepared by adding 2.5 grams of gelatin, 16.3 grams of chrom-alum, 34.7 grams of methanol, 12.7 grams of sodium silicate in 33.9 grams of distilled water.

Control: Formulations 1-1 was coated on a glass plate using the coating device. The formulations were introduced through a slot-coating die at a temperature of 45 degrees C onto a 20.3 cm wide substrate moving at the rate of 3.1 m/min. The flow rate was adjusted to provide a level of 86.1 -g/m2 gelatin coverage. The coatings were chill-set in a 9.1 meter long chilling section that was maintained at a temperature of 4 degrees C and 56.6% RH and then dried in 3 drying sections that totaled 34 meters at a temperature and RH of 35 degrees C and 18.3% RH respectively.

Invention: Formulation 1-2 is added to a pan at a temperature of 45 degrees C. The solution is contacted by roll whereby it is transferred to Gravure roll. It is skived off to obtain the desired thickness. The solution is then picked up by a roll whereby it comes in contact with the substrate. A thin layer of solution is deposited on the substrate. The substrate is conveyed through a I 0-meter dryer section at a temperature of 51.6 degrees C. This solution is applied to the topside of the substrate and acts as an adhesion layer for Formulation 1-1.

The invention produces satisfactory coating with upper gelatin layer tightly bound to the glass during wet processing, in contrast, the gelatin layer frills during the drying process.

### Example 2

This example illustrates the method of evaluating gelatin coated protein microarray substrate using a modified enzyme linked immunosobent assay (ELISA).

The procedure to perform the modified ELISA is follows.

1. Goat anti-mouse antibody IgG from Sigma was dissolved in PBS (phosphate saline buffer, pH7.4) buffer to a concentration of 1 mg/mL. A series of diluted of goat anti-mouse antibody IgG were spotted manually onto nitrocellulose membrane and coated gelatin substrates. The spotted substrates were incubated in a humid chamber for 1 hour at room temperature.

2. The substrates were washed four times in PBS buffer with 1% Triton X100™, 5 min each time with shaking.

3. The washed substrates were incubated in PBS buffer with 1% glycine for 15 min with constant shaking.

4. The substrates were washed four times in PBS buffer with 1% Triton™ X100 with shaking.

5. Mouse IgG from Sigma was diluted in PBS buffer with 0.1% Tween™ 20 to 1 μg/mL to cover the whole surface of substrates, and the substrates were incubate at room temperature for 1 hour.

6. The substrates were washed four times with PBS buffer with 1% Triton X100, 5 min each time with constant shaking.

7. The substrates were incubated in goat anti-mouse IgG horse radish peroxidase conjugate (diluted in PBS with 1% glycine to appropriate titer) solution to cover the whole surface of the substrates at room temperature for 1 hour with shaking.

8. The substrates were washed four times with PBS buffer with 1% Triton X100, 5 min each time with constant shaking, and rinsed twice in water.

9. The color were developed in horse radish peroxidase substrate solution containing SuperSignal® ELISA chemiluminescence substrate solution (purchased from PIERCE ENDOGEN). The chemiluminescence image was capture by contacting a thin layer of SuperSignal® ELISA chemiluminescence substrate solution (purchased from PIERCE ENDOGEN) with coated substrate. The emission was measured on Kodak Image Station 440 and quantified using Region of Interest (ROI) software supplied with the instrument.

1. A protein microarray element comprising:
a) a support;
b) a gelatin layer containing functional groups capable of binding biological probes; and interposed between the support and the gelatin layer c) an adhesive interlayer layer capable of maintaining contact with the support and with the gelatin layer.

2. The microarray of claim 1 wherein the support is organic or inorganic.

3. The microarray of claim 1 wherein the support is glass or fused silica.

4. The microarray of claim 1 wherein the support is between 0.1 and 5.0 mm in thickness.

5. The microarray of claim 1 wherein the support is between 0.5 and 2.0 mm in thickness.

6. The microarray of claim 1 wherein the adhesive interlayer comprises proteins, protein derivatives, gelatin, gelatin derivatives, or hydrophilic water-permeable colloids.

7. The microarray of claim 1 wherein the adhesive interlayer layer comprises synthetic polymeric peptizers, carriers, or binders.

8. The microarray of claim 1 wherein the adhesive interlayer layer comprises poly(vinyl alcohol), poly(vinyl lactams), acrylamide polymers, polyvinyl acetals, polymers of alkyl and sulfoalkyl acrylates and methacrylates, hydrolyzed polyvinyl acetates, polyamides, polyvinyl pyridine, or methacrylamide copolymers.

9. The microarray of claim 1 wherein the adhesive interlayer layer comprises gelatin.

10. The microarray of claim 9 wherein an organic solvent, or a mixture of solvents is combined with the gelatin.

11. The microarray of claim 10 wherein the organic solvent or mixture of solvents includes acetone, alcohol, ethyl acetate, methylene chloride, ether, or a mixture thereof.

12. The microarray of claim 9 wherein a crosslinking agent, a silicate salt, or a dispersing aid is combined with the gelatin.

13. The microarray of claim 9 wherein the gelatin is alkaline pretreated.

14. The microarray of claim 9 wherein the gelatin is pig gelatin or fish gelatin.

15. The microarray of claim 9 wherein the gelatin coverage is 0.2 to 100 grams per square meter.

16. The microarray of claim 9 wherein the gelatin coverage is 10 to 50 grams.

17. A protein microarray element comprising:
a) a support;
b) on said support is disposed an adhesive layer capable of maintaining contact with the support; and with
c) a gelatin layer that bears a trifunctional compound A-L-B; wherein A is a functional group capable of interacting with the gelatin; L is a linking group capable of interacting with A and with B; and B is a functional group capable of interacting with a protein capture agent; wherein A may be the same or different from B.

18. The microarray of claim 17 wherein the trifunctional compound A-L-B is a polymer scaffold affixed to said gelatin layer.

19. The microarray of claim 17 wherein the polymer in the polymer scaffold is rich in reactive units capable of interacting with proteins.

20. The microarray of claim 17 wherein the interaction between the gelatin layer and A is a physical binding or a chemical reaction.

21. The microarray of claim 17 wherein either A or B, or both, is aldehyde, epoxy, hydrazide, vinyl sulfone, succinimidyl ester, carbodiimide, maleimide, dithio, iodoacetyl, isocyanate, isothiocyanate, or aziridine.

22. The microarray of claim 17 wherein B is an affinity tag capable of interacting non-covalently with a protein capture agent that is to be immobilized onto the substrate.

23. A method of making a gelatin-based substrate for fabricating protein arrays, the method comprising the steps of:
providing a support;
coating on the support an adhesion layer;
coating, on said adhesion layer, a layer of gelatin containing a trifunctional compound A-L-B; wherein A is a functional group capable of interacting with the gelatin; L is a linking group capable if interacting with A and with B; and B is a functional group capable of interacting with a protein capture agent;
wherein A may be the same or different from B.

24. The method of claim 23 wherein the trifunctional compound ALB is affixed while coating the gelatin on the adhesion layer.

25. The method of claim 23 wherein the trifunctional compound ALB is affixed after coating the gelatin on the adhesion layer.

26. The method of claim 23 wherein the protein capture agent is antibody, protein scaffold, peptide, nucleic acid ligand, or a molecular imprinting polymer.

27. The method of claim 23 wherein the polymer in the polymer scaffold is rich in reactive units that are capable of immobilizing proteins.
